(19) United States
(12) Patent Application Publication
(10) Pub. No.: US 2013/0079644 A1
(43) Pub. Date: Mar. 28, 2013

(54) Optical Probe with Electric Motor

(71) Applicants: Jan Peeters Weem, Forest Grove, OR (US); Laurence A. Daane, Portland, OR (US); Jason M. Woods, Portland, OR (US)

(72) Inventors: Jan Peeters Weem, Forest Grove, OR (US); Laurence A. Daane, Portland, OR (US); Jason M. Woods, Portland, OR (US)

(73) Assignee: Tyco Electronics Corporation, Berwyn, PA (US)

(21) Appl. No.: 13/243,922

(22) Filed: Sep. 23, 2011

(51) Int. Cl. A61B 6/00; G01N 21/55

(52) U.S. Cl. 600/476; 356/445

(57) ABSTRACT

Optical probes may be used to capture images of a subject. An optical probe may include an optical reflector, an optical waveguide, and an electric motor. A rotor of the electric motor is mechanically coupled with the optical reflector to rotate the optical reflector in response to an input electric current. The optical waveguide is optically coupled with the optical reflector. The optical waveguide may output light to the optical reflector which directs the light to the subject. Light may then reflect back from the subject to the optical reflector which directs the reflected light from the subject to the optical waveguide.

### BACKGROUND

1. Technical Field

This application relates to imaging systems and, more particularly, to optical probes.

2. Related Art

Optical probes are often used to capture images of subjects that may be hidden from open view. For example, a physician may guide an optical probe into a bodily lumen, such as a blood vessel, to capture images of blockages, occlusions, plaques, or other subjects within the vessel. One imaging technique that uses optical probes is Optical Coherence Tomography (“OCT”). In OCT, a light source sends light waves through an optical waveguide, such as an optical fiber. The light waves are output from the optical fiber and directed against the subject to be imaged. At least some of the light reflects off the subject and is captured by optical fiber. The light reflected off the subject is then analyzed to create an image of the subject.

Some optical imaging probes are designed in a “side-viewing” implementation. These probes are helpful when the area to be imaged is positioned on a side of the probe rather than in-line with the end of the probe. For example, an OCT optical probe may direct light against a side wall of a blood vessel to analyze the plaque on the sides of the vessel wall as the probe is guided through the vessel. Some imaging systems apply torque to a portion of the optical probe to change a direction of the light output from the side of the probe. For example, a physician in an OCT procedure may rotate the proximal end of the probe to change the direction of the light output from the distal end of the probe to create a 360 degree image of a portion of a vessel wall.

In some situations, rotation of the optical probe may cause non-uniform rotational distortion (“NURD”) problems. For example, mechanical drag on various portions of the probe may result when the optical probe is rotating in a space with a small diameter or several curves. The mechanical drag causes some portions of the probe to rotate differently than other portions of the probe. This non-uniform rotation may lead to significant distortions and artifacts in the images captured by the rotating optical probe. Thus, a need exists for an optical probe that more resistant to rotational distortion effects.

### SUMMARY

Optical probes may be used to capture images of a subject. In one implementation, an optical probe includes an optical reflector, an electric motor, and an optical waveguide. The electric motor includes a rotor that is mechanically coupled with the optical reflector. The optical waveguide is optically coupled with the optical reflector.

In another implementation, the optical probe includes an optical reflector mechanically coupled with an electric motor. The electric motor comprises a motor shaft that defines an opening for an optical waveguide to transmit light through the electric motor to the optical reflector. The electric motor is configured to rotate the optical reflector about an axis of the motor shaft.

In yet another implementation, the optical probe includes an optical reflector, a motor shaft, a permanent magnet, and a coil. The motor shaft defines an opening for an optical waveguide to transmit light through the motor shaft to the optical reflector. The permanent magnet is mechanically coupled with the optical reflector. The coil is positioned relative to the permanent magnet so that a magnetic field generated in response to an input electric current passing through the coil causes rotation of the permanent magnet and the optical reflector about the motor shaft.

Other systems, methods, features and advantages will be, or will become, apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

FIG. 1 illustrates an optical probe system.

FIG. 2 illustrates an implementation of an optical probe with a rotatable motor shaft.

FIG. 3 illustrates another implementation of an optical probe with a rotatable motor shaft.

FIG. 4 illustrates yet another implementation of an optical probe with a rotatable motor shaft.

FIG. 5 illustrates an optical probe with a fixed motor shaft.

FIG. 6 illustrates a three-dimensional view of an optical probe with a fixed motor shaft.

FIG. 7 illustrates a flat mirror optical reflector of an optical probe.

FIG. 8 illustrates a prism optical reflector of an optical probe.

FIG. 9 illustrates a curved mirror optical reflector of an optical probe.

FIG. 10 illustrates a three-dimensional view of an optical probe with a rotatable motor shaft.

### DETAILED DESCRIPTION OF THE PREFERRED EMBODIMENTS

An optical probe system may be used to capture images of a subject. For example, a physician may guide an optical probe into a bodily lumen, such as a blood vessel, to capture images of blockages, occlusions, plaques, or other subjects within the vessel. The optical probe may direct light against the subject and capture light reflected back from the subject. The light reflected off the subject is analyzed to create an image of the subject. Optical probes may also be used to perform other functions, such as data communication through optical fibers.

The optical probes described herein include an electric motor that rotates a portion of the probe to change a direction of the light output from the probe. In implementations where the electric motor of the probe rotates only a sub-portion of the light path through the probe while leaving other portions of the light path stationary, some of the problems associated with non-uniform rotational distortion (“NURD”) may be avoided. For example, the electric motor may be configured to rotate only a light emitting/capturing distal end portion of the probe while leaving all or a majority of the optical waveguide through the probe substantially stationary. In this configuration, the substantially static optical waveguide that carries the light along the length of the probe would not experience the rotational irregularities seen in dynamically rotated waveguides, which may improve the performance of the probe.

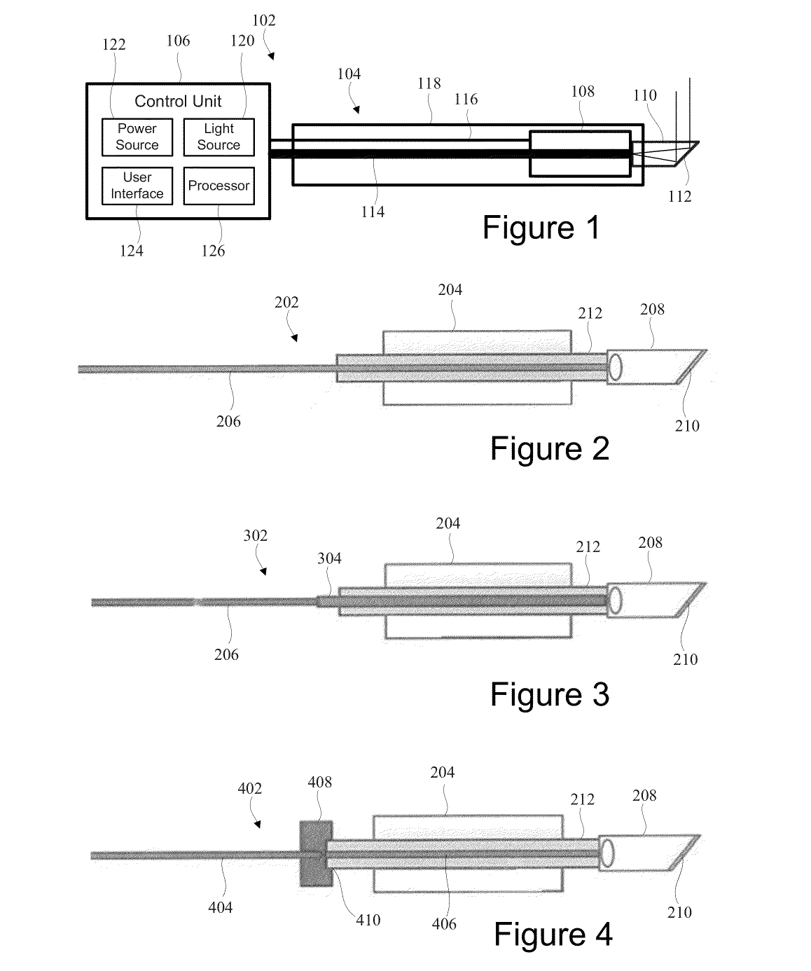

FIG. 1 illustrates an optical probe system 102. The system 102 includes an optical probe 104 and a control unit 106. The control unit 106 allows a user to control the supply of power and light to the probe 104. In one implementation, the probe 104 delivers the light against the imaging subject, captures light reflected back from the subject, and delivers the light reflected from the subject to the control unit 106 for image processing. The optical probe 104 includes a proximal end and a distal end. In the perspective of FIG. 1, the proximal end of the probe 104 is the portion of the probe 104 that is closest to the control unit 106 and the distal end of the probe 104 is the portion of the probe 104 that is furthest from the control unit 106.

In the implementation of FIG. 1, the probe 104 includes an electric motor 108, a distal tip 110, an optical reflector 112, an optical waveguide 114, a power supply link 116, and an outer sheath 118. The outer sheath 118 may be a catheter housing through which the other components of the probe 104 may pass. For example, the outer sheath 118 may be placed into a body lumen, such as a blood vessel, to act as a guide for the remainder of the probe 104 to be inserted into or removed from the body lumen. In one implementation, such as for an Optical Coherence Tomography (“OCT”) procedure, the diameter of the probe 104 may be in the range of about 3-9 French (1-3 mm). In other implementations, other probe dimensions may be used.

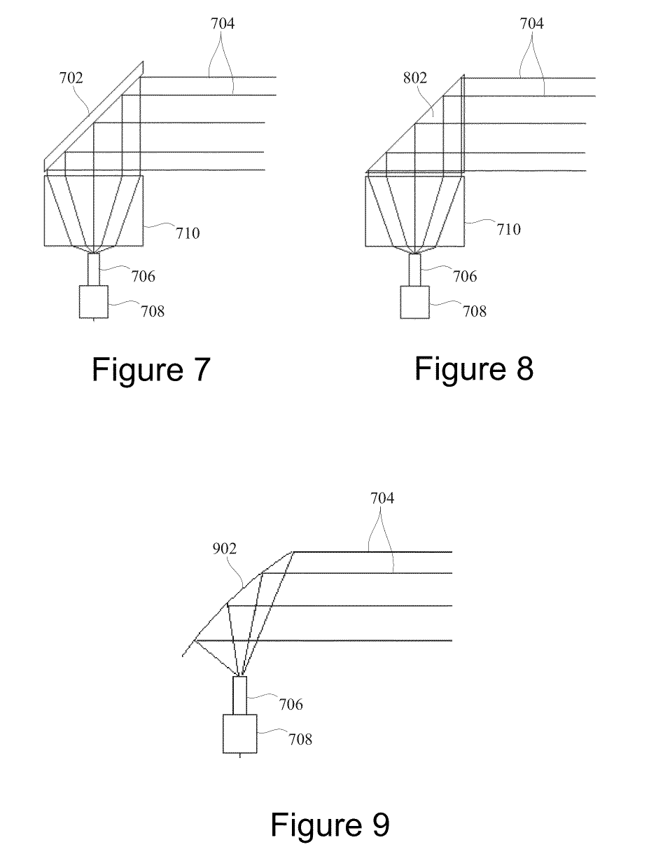

The distal tip 110 in one implementation is a catheter housing portion at the distal end of the probe 104. The distal tip 110 rotates with the rotor of the electric motor 108. The optical reflector 112 may be mechanically coupled with the distal tip 110 so that the optical reflector 112 rotates with the distal tip 110. For example, the optical reflector 112 may be connected with a housing component of the distal tip 110. The distal tip 110 in the implementation of FIG. 1 rotates with the rotor of the electric motor 108 to change the direction of the light output from the probe 104. Light is emitted from the optical waveguide 114 onto the optical reflector 112 and is output from a side of the probe 104 to achieve a “side-viewing” optical probe implementation. FIGS. 7-9, described below, show various implementations of optical reflectors 112 that change the direction of light to achieve the “side-viewing” optical probe implementation. In an implementation where the probe 104 is used to capture images of plaque inside a blood vessel, the rotation of the optical reflector 112 allows the probe to capture images of multiple sides of the blood vessel, such as a 360 degree view around an inner surface of a section of the blood vessel. As the optical reflector 112 rotates the probe 104 captures images of different portions of the vessel wall.

The electric motor 108 is mechanically coupled with the optical reflector 112 so that the motor 108 may rotate the optical reflector 112 in response to an input electric current to the motor 108. The input electric current passes from the control unit 106 through the power supply link 116 to the electric motor 108. The power supply link 116 may include one or more power supply lines between the control unit 106 and the electric motor 108.

In one implementation, the electric motor 108 may include a rotor coupled with the optical reflector 112. The rotor may be coupled with the optical reflector 112 by either a direct or indirect connection. In one implementation, the rotor is connected with a housing component, such as the distal tip 110 of the probe 104, which is connected with the optical reflector 112. In this implementation, rotation of the rotor causes rotation of the housing component, which causes rotation of the optical reflector 112. The electric motor 108 may be a brushed motor, brushless motor, direct current motor, alternating current motor, stepper motor, or another device that converts electrical energy into mechanical energy. In one implementation, the motor may be a modified version of a small diameter micro geared motor, such as the 1.5 mm diameter micro-motor available from the Namiki Precision Jewel Co., Ltd. For example, a general purpose motor may be modified to have a hollow shaft sized to allow passage of the optical waveguide 114.

The electric motor 108 may define an opening for the optical waveguide 114 to transmit light through at least a portion of the electric motor 108 to the optical reflector 112. In one implementation, the electric motor 108 may include a hollow motor shaft with a passageway through the hollow center of the motor shaft that allows passage of the optical waveguide 114. The opening through the electric motor 108 allows the optical waveguide 114 to pass through the motor 108 so that the optical waveguide 114 can be optically coupled with the optical reflector 112. The optical waveguide 114 and the optical reflector 112 are optically coupled in configurations where light output from the optical waveguide 114 is able to reach the optical reflector 112. The optical waveguide 114 and the optical reflector 112 are also optically coupled in configurations where light from the optical reflector 112 is able to reach the optical waveguide 114. The optical coupling may be achieved directly, such as through an air or vacuum medium, or indirectly, such as through a lens or other optical coupling device. In one implementation, the optical waveguide 114 is optically coupled with the optical reflector 112 in a manner that allows rotation of the optical reflector 112 without corresponding rotation of the optical waveguide 114. For example, the optical waveguide 114 may remain stationary while the optical reflector 112 rotates to change the output direction of light from the probe 104.

The optical waveguide 114 comprises a medium that guides electromagnetic waves in the optical spectrum. In one implementation, the optical waveguide comprises a physical structure, such as an optical fiber. The optical fiber may be formed from a glass, polymer, or semiconductor. The optical waveguide 114 may pass all the way through the electric motor 108 (e.g., along the entire length of a hollow motor shaft) or may pass through only a sub-portion of the electric motor 108 (e.g., along only a sub-portion of the entire length of a hollow motor shaft).

In the implementation of FIG. 1, the control unit 106 includes a light source 120, a power source 122, a user interface 124, and a processor 126. The light source 120 transmits light through the optical waveguide 114 of the probe 104 for use in the imaging process. The light source 120 may be a superluminescent diode, pulsed laser, tunable laser, or other type of light source. In one implementation where the optical probe is configured for Optical Coherence Tomography, the light source 120 may emit light waves with wavelengths of about 1300 nm. The light source 120 may also produce light waves with other wavelengths or light characteristics in other implementations.

The power source 122 supplies electrical current to the probe 104. For example, the electrical current from the power source 122 may be passed through the power source link 116 to drive the electric motor 108 of the probe 104. The power source 122 may be a direct current (DC) power supply or an alternating current (AC) power supply.

The user interface 124 provides a user of the optical probe system 102 with control over the rotation of the optical reflector 112 of the optical probe 104. For example, the user interface 124 may include a switch, dial, graphical user interface, or other rotation control mechanism. In one implementation, the user interface 124 comprises an “on/off” switch that either drives the motor at one speed or leaves the motor in an off state. In another implementation, the user interface 124 allows for a motor speed selection capability, such as through a variable position dial, analog voltage supplier, or processor-controlled user interface. The user interface 124 may control the rotation speed of the motor 108 by controlling the amount of electric current passed to the motor 108.

The processor 126 may control the delivery of power to the probe 104, control the delivery of light to the probe 104, and/or perform image processing. For example, the processor 126 may analyze data related to the light received back from the probe 104 to create an image. Also, the processor 126 may control how the motor of the probe 104 is driven, such as by creating power pulse sequences to achieve the desired rotation characteristics.

FIG. 2 illustrates one implementation of an optical probe 202 with an electric motor 204. The optical probe 202 includes an optical waveguide 206, a distal tip 208, and an optical reflector 210, which may be the same or similar to the corresponding components of the optical probe 104 of FIG. 1. The optical probe 202 of FIG. 2 illustrates one implementation of an electric motor where the rotor of the electric motor 204 is a hollow motor shaft 212. The motor 204 rotates the hollow motor shaft 212 about a longitudinal axis of the hollow motor shaft 212 in response to an input electric current. This rotation of the hollow motor shaft 212 serves to rotate the optical reflector 210. For example, the hollow motor shaft 212 may be coupled with the optical reflector 210, either directly or through the housing of the distal tip 208. The hollow motor shaft 212 defines an opening for the optical waveguide 206 to transmit light through the electric motor 204 to the optical reflector 112. The hollow center of the hollow motor shaft 212 allows light from the optical waveguide 206 to reach the optical reflector 112 with minimal interference from the structure of the motor 204. For example, the light can pass through the motor 204 without components of the motor blocking any portion of the light path.

FIG. 3 illustrates another implementation of an optical probe 302 with an electric motor 204. The optical probe 302 of FIG. 3 is the same as the optical probe 202 of FIG. 2 except for the addition of a protective material 302 disposed between the optical waveguide 206 and the hollow motor shaft 212. The protective material 302 serves to shield the optical waveguide 304 from damage. For example, without the protective material 302, the optical waveguide 206 may be subject to abrasion due to contact between the rotating motor shaft 212 and the stationary optical waveguide 206. In one implementation, the protective material 302 is coupled with an outer coating of the optical waveguide 206 as a secondary coating. In another implementation, the protective material 302 is coupled with an inner surface of the hollow motor shaft 212. The protective material 302 may be a bearing, bushing, gel, lubricant, polymer (e.g., fluoropolymer heat shrink), or another extra coating that protects the optical waveguide 206 from damage due to rotation of the hollow motor shaft 212.

FIG. 4 illustrates another implementation of an optical probe 402 with an electric motor 204. The construction and operation of the electric motor 204, the distal tip 208, the optical reflector 210, and the hollow motor shaft 212 in the optical probe 402 of FIG. 4 may be the same as in the optical probes 202 and 302 of FIGS. 2 and 3. The optical probe 402 of FIG. 4 differs from the optical probes 202 and 302 of FIGS. 2 and 3 in that the optical probe 402 of FIG. 4 includes two separate optical waveguides 404 and 406 coupled together via an optical connector 408. The optical connector 408 optically couples an optical path of the optical waveguide 404 with an optical path of the optical waveguide 406 so that light output from one on the waveguides is aligned with an input of the other waveguide. For example, the optical connector 408 may include a lens system that directs light waves between the corresponding ends of the waveguides 404 and 406.

The use of two separate waveguides allows one of the waveguides to be stationary while the other of the waveguides rotates. In the implementation of FIG. 4, the optical waveguide 404 is held stationary while the optical waveguide 406 rotates with the motor shaft 212. For example, the optical waveguide 406 may be connected with an interior surface of the hollow motor shaft 212. The optical waveguide 404 is physically separate from the optical waveguide 406 in a configuration where the optical waveguide 406 rotates with the motor shaft 212 without corresponding rotation of the second optical waveguide.

The optical waveguide 406 in one implementation may be an optical fiber that guides light through an open core/shaft of a motor so that the light reaches the output tip of the system. In another implementation, the optical waveguide 406 may be an optically clear motor core/shaft that allows light transmission. For example, the optical waveguide 406 may be an optically clear portion of the motor shaft that is optically coupled with another optical waveguide 404. Another waveguide, such as an optical fiber, may then direct light to the optically clear core/shaft. Thus, the optically clear core/shaft (e.g., the waveguide 406) could rotate with the motion of the motor while leaving the other optical waveguide (e.g., the waveguide 404) substantially stationary.

The optical connector 408 serves to align the end of one waveguide with the end of another waveguide so that light may pass between the waveguides. In one implementation, the optical connector 408 includes a notch 410 in a portion of the optical connector 408 sized to receive a proximal end portion of the hollow motor shaft 212. The notch 410 is positioned to hold the optical path of the optical waveguide 406 in alignment with the optical path of the optical waveguide 404 during rotation of the hollow motor shaft 212.

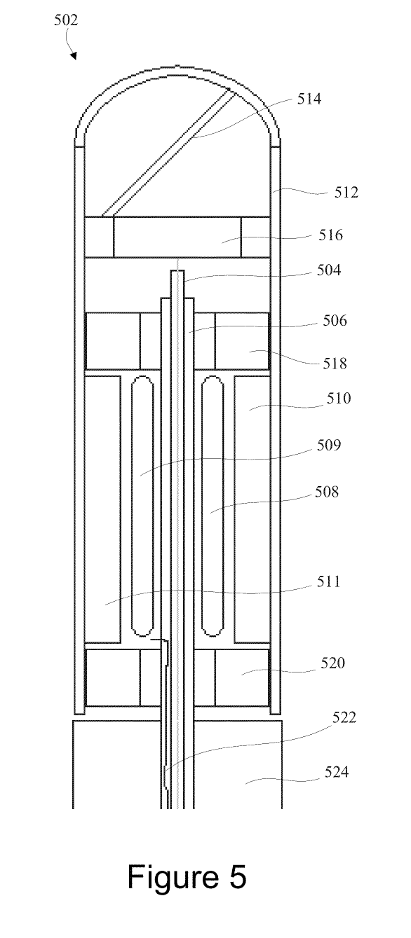

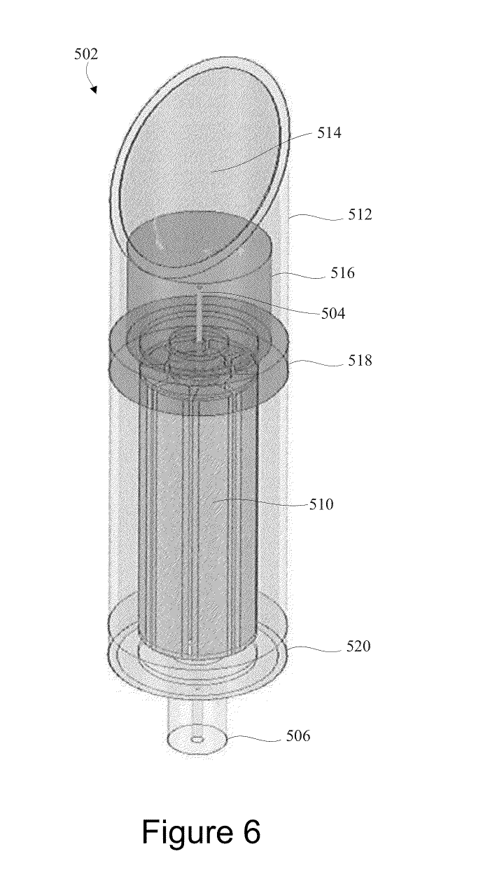

FIG. 5 illustrates another implementation of an optical probe 502 with an electric motor, such as a brushless direct current spindle motor. FIG. 6 illustrates a three-dimensional view of the optical probe of FIG. 5. The optical probe 502 includes an optical waveguide 504, a motor shaft 506, coils 508 and 509, permanent magnets 510 and 511, a distal tip catheter housing 512, an optical reflector 514, a lens 516, rotation guide components 518 and 520, a power supply link 522, and a catheter body 524 disposed on a proximal end of the rotating distal tip of the probe 502.

The optical waveguide 504 passes through an opening in the motor shaft 506 so that light may pass through the motor of the probe 502 and reach the optical reflector 514. The motor shaft 506 in the optical probe 502 may be stationary. For example, the motor of the optical probe 502 does not rotate the motor shaft 506. Rather, the rotor of the motor rotates about the motor shaft 506.

The electric motor of the optical probe 502 includes the coils 508 and 509, and the permanent magnets 510 and 511. In the implementation of FIG. 5, the coils 508 and 509 serve as the stators of the electric motor and the permanent magnets 510 and 511 serve as the rotors of the electric motor. The coils 508 and 509 may be formed from a conductive wire, such as copper or another high conductivity alloy, into a cylindrical coil shape. The coils 508 and 509 may include a ferromagnetic core or may have an air core.

The optical probe 502 in FIG. 5 illustrates two coils and two magnets. Other implementations may include more than two coils and more than two magnets. The magnets of the motor may be disposed around the motor shaft 506 and coupled with the distal tip catheter housing 512 to provide rotational force to the distal tip catheter housing 512. The coils of the motor may be disposed about the motor shaft 506 between an outer surface of the motor shaft 506 and the magnets. The coils may be physically connected to the motor shaft 506, via solder, epoxy, clamp, crimp, or another connection mechanism.

The power supply link 522 may pass through at least a portion of the motor shaft 506. In one implementation, the motor shaft 506 defines an opening on a side of the shaft to allow the one or more lines of the power supply link 522 to exit the hollow center of the motor shaft 522 and connect with the motor of the probe 502. The power supply link 522 may comprise one or more power supply lines that provide electric current to the coils 508 and 509. For example, the power supply link 522 may include a first power line to the coil 508 and a second power line to the coil 509. The control unit that provides power to the power lines of the power supply link 522 may stagger the application of electric current to the power lines to provide a rotational movement of the permanent magnets 510 and 511 about the motor shaft 506. For example, the control unit may pulse each of the coils 508 and 509 out of phase with each other so that the coils 508 and 509 cause the magnets 510 and 511 to rotate. Specifically, the coils 508 and 509 generate magnetic fields when an input electric current is passing through the coils 508 and 509. The coils 508 and 509 are positioned to be near the permanent magnets so that an interaction between the magnetic fields and the permanent magnets 510 and 511 is strong enough to cause rotation of the permanent magnets 510 and 511 about the motor shaft 506.

The permanent magnets 510 and 511 of the probe 502 are coupled with the distal tip catheter housing 512. The distal tip catheter housing 512 is coupled with the optical reflector 514. Thus, the permanent magnets 510 and 511, which serve as rotors of an electric motor of the optical probe 502, are coupled with the optical reflector 514 and can cause rotation of the distal tip catheter housing 512 and the optical reflector 514 about a longitudinal axis of the motor shaft 506.

The rotation guide components 518 and 520 may be disposed between the rotating portion of the optical probe 502 and the stationary motor shaft 506. The rotation guide components 518 and 520 allow rotation around the motor shaft 506 with reduced friction and increased stability. In one implementation, the rotation guide components 518 and 520 may be disposed between the distal tip catheter housing 512 and the motor shaft 506. In another implementation, the rotation guide components 518 and 520 may be disposed between the motor shaft 506 and the rotating magnets 510 and 511. The rotation guide components 518 and 520 may be bearings, bushings, or other devices to guide the rotation of the magnets about the motor shaft 506. The rotation guide components 518 and 520 may provide a slip coupling between the rotating portion of the optical probe 502 and the stationary motor shaft 506. For example, the rotation guide components 518 and 520 may rotate with the magnets 510 and 511, the distal tip catheter housing 512, and the optical reflector 514 while sliding along a surface of the motor shaft 506. In one implementation, the rotation guide components 518 and 520 may be formed in a disk shape with a hole that fits around the motor shaft 506.

FIG. 7 illustrates a flat mirror optical reflector 702 of an optical probe. The flat minor optical reflector 702 may be positioned in a distal end portion of the optical probe. The flat minor optical reflector 702 serves to change the direction of the light waves 704 emitted from (and/or reflected back to) an optical waveguide 706. In the implementation of FIG. 7, the flat minor optical reflector 702 is positioned on an angle to change the light direction by 90 degrees. In other implementations, the flat mirror optical reflector 702 may change the light direction by other amounts, such as by greater than 0 degrees or less than 180 degrees.

In the implementation of FIG. 7, the optical waveguide 706 passes through a hollow motor shaft 708. Also, the implementation of FIG. 7 includes a lens 710 disposed in a light path between the optical waveguide 706 and the flat minor optical reflector 702. The lens 710 may be a graded index (“GRIN”) lens or any other type of lens that focuses and/or aligns light between the flat mirror optical reflector 702 and the end of the optical waveguide 706. In one implementation, the lens 710 is a collimating lens. A collimating lens may cause the light beams passing through the lens to become more aligned in a specific direction (e.g., parallel or substantially parallel). The lens 710 in FIG. 7 is shown to spread the light output from the optical waveguide so that the light waves are parallel or substantially parallel when output from the lens 710. The lens 710 also serves to narrow the light reflected from the imaging subject to focus the reflected light to the end of the optical waveguide 706. The lens 710 may be fused directly to the end of the optical waveguide 706 or may be separated from the end of the optical waveguide 706 by an air gap. The gap between the lens 710 and the optical waveguide 706 may also include an optical index matching compound to reduce the reflections of light at the interface of the two components.

FIG. 8 illustrates a prism optical reflector 802 of an optical probe. The prism optical reflector 802 may be positioned in a distal end portion of the optical probe. The prism optical reflector 802 serves to change the direction of the light waves 704 emitted from (and/or reflected back to) an optical waveguide 706, such as through total internal reflection. In the implementation of FIG. 8, the optical waveguide 706 passes through a hollow motor shaft 708. Also, the implementation of FIG. 8 includes a lens 710 disposed in a light path between the optical waveguide 706 and the prism optical reflector 802. The lens 710 and the prism optical reflector 802 may be separate components, joined together, or constituent portions of one component. In one implementation, the prism optical reflector 802 may be a 3 mm right angle prism available from Thorlabs, Inc. In other embodiments, other prism shapes and sizes may be used.

FIG. 9 illustrates a curved mirror optical reflector 902 of an optical probe. The curved minor optical reflector 902 may be positioned in a distal end portion of the optical probe. The curved mirror optical reflector 902 serves to change the direction of the light waves 704 emitted from (and/or reflected back to) an optical waveguide 706. In the implementation of FIG. 8, the optical waveguide 706 passes through a hollow motor shaft 708. The curvature shape of the curved minor optical reflector 902 in the implementation of FIG. 8 may replace the need for the lens 710. For example, the curvature shape may be designed to both collimate the light and direct it to the side of the probe (or back from the side of the probe to the end of the optical waveguide 706).

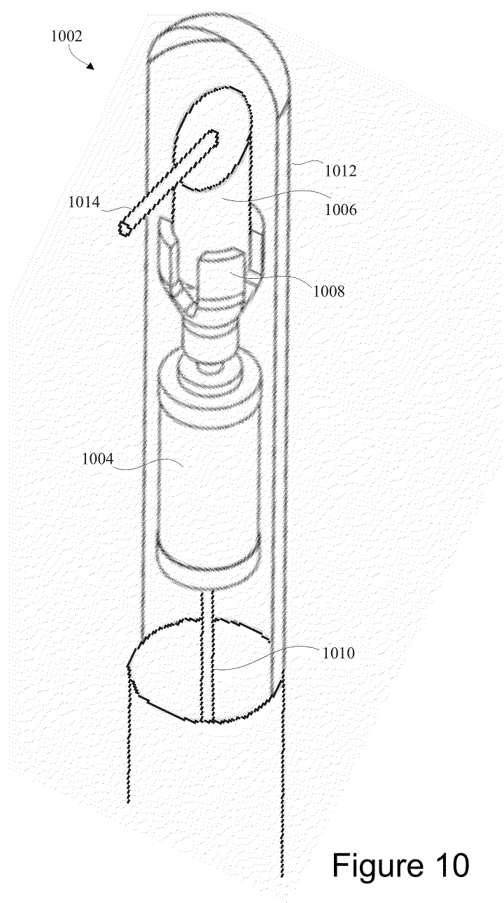

FIG. 10 illustrates a three-dimensional view of an optical probe 1002 with a rotatable motor shaft. The optical probe 1002 includes an electric motor 1004, an optical reflector 1006, a connection component 1008, an optical waveguide 1010, and an outer sheath 1012. The connection component 1008 is coupled between the optical reflector 1006 and a motor shaft of the electric motor 1004. The connection component 1008 and the optical reflector 1006 may rotate with the motor shaft to change an output direction of the light 1014.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

What is claimed is:

1. An optical probe, comprising:
an optical reflector;
an electric motor with a rotor mechanically coupled with the optical reflector; and
an optical waveguide optically coupled with the optical reflector.

2. The optical probe of claim 1, wherein the electric motor defines an opening that allows passage of the optical waveguide through at least a portion of the electric motor.

3. The optical probe of claim 1, wherein the rotor comprises a hollow motor shaft, wherein the electric motor is configured to rotate the hollow motor shaft and the optical reflector about a longitudinal axis of the hollow motor shaft in response to an input electric current.

4. The optical probe of claim 1, wherein the rotor comprises a hollow motor shaft, wherein the optical waveguide comprises an optical fiber that passes through at least a portion of the hollow motor shaft.

5. The optical probe of claim 4, wherein the optical reflector is optically coupled with the optical fiber in a configuration that allows rotation of the optical reflector without corresponding rotation of the optical fiber.

6. The optical probe of claim 4, further comprising a bearing, bushing, or protective material disposed between the optical fiber and the hollow motor shaft.

7. The optical probe of claim 1, wherein the optical waveguide is a first optical waveguide, the optical probe further comprising:
a second optical waveguide; and
an optical connector that optically couples an optical path of the first optical waveguide with an optical path of the second optical waveguide;
wherein the first optical waveguide is connected with the rotor and rotates with the rotor without corresponding rotation of the second optical waveguide.

8. The optical probe of claim 7, wherein the optical connector comprises a notch sized to receive a proximal end portion of the rotor, and wherein the notch is positioned to hold the optical path of the first optical waveguide in alignment with the optical path of the second optical waveguide during rotation of the rotor.

9. The optical probe of claim 1, wherein the rotor comprises a rotor magnet, the optical probe further comprising:
a stator coil configured to generate a magnetic field in response to an electric current passing through the stator coil; and
a motor shaft;
wherein the stator coil is positioned relative to the rotor magnet so that the rotor magnet and the optical reflector rotate about the motor shaft in response to the rotor magnet experiencing the magnetic field.

10. The optical probe of claim 1, wherein the optical reflector comprises a flat mirror or prism positioned relative to the optical waveguide to change a direction of light output from the optical waveguide.

11. The optical probe of claim 1, further comprising a lens disposed between the optical reflector and a distal end of the optical waveguide.

12. The optical probe of claim 1, wherein the optical reflector comprises a shaped mirror having a curvature that collimates and changes a direction of light output from the optical waveguide.

13. The optical probe of claim 1, wherein the electric motor comprises a brushless direct current spindle motor.

14. The optical probe of claim 1, wherein the optical waveguide comprises an optically clear core or shaft of the electric motor.

15. An optical probe, comprising:
an optical reflector; and
an electric motor mechanically coupled with the optical reflector;
wherein the electric motor comprises a motor shaft that defines an opening for an optical waveguide to transmit light through at least a portion of the electric motor to the optical reflector, and wherein the electric motor is configured to rotate the optical reflector about an axis of the motor shaft.

16. The optical probe of claim 15, wherein the optical reflector is optically coupled with the optical waveguide in a manner that allows rotation of the optical reflector without corresponding rotation of the optical waveguide.

17. The optical probe of claim 15, wherein the electric motor comprises:
a stator coil configured to generate a magnetic field in response to an electric current passing through the stator coil; and
a rotor magnet mechanically coupled with the optical reflector;
wherein the stator coil is positioned relative to the rotor magnet so that the rotor magnet and the optical reflector rotate about the motor shaft in response to the rotor magnet experiencing the magnetic field.

18. An optical probe, comprising:
an optical reflector;
a motor shaft that defines an opening for an optical waveguide to transmit light through at least a portion of the motor shaft to the optical reflector;
a permanent magnet mechanically coupled with the optical reflector; and
a coil positioned relative to the permanent magnet so that a magnetic field generated in response to an input electric current passing through the coil causes rotation of the permanent magnet and the optical reflector about the motor shaft.

19. The optical probe of claim 18, wherein the optical reflector is optically coupled with the optical waveguide in a manner that allows rotation of the optical reflector without corresponding rotation of the optical waveguide.

20. The optical probe of claim 18, further comprising:
a distal tip catheter housing mechanically coupled with the optical reflector; and
a rotation guide component mechanically coupled between the distal tip catheter housing and the motor shaft.

21. The optical probe of claim 18, wherein the coil is a first coil and the permanent magnet is a first magnet;
wherein the optical probe further comprises a plurality of second coils and a plurality of second magnets;
wherein the first magnet and the plurality of second magnets are mechanically coupled with a distal tip catheter housing and are disposed about the motor shaft; and
wherein the first coil and the plurality of second coils are disposed about the motor shaft between an outer surface of the motor shaft, and the first magnet and the plurality of second magnets.
